(19) United States
(12) Patent Application Publication
(10) Pub. No.: US 2004/0009596 A1
(43) Pub. Date: Jan. 15, 2004

(54) Extract from an Indian green mussel (perna viridis) for differentiation and maturation of dendric cells

(76) Inventors: Kanury V. S. Rao, New Delhi (IN); Krishnamurthy Natarajan, New Delhi (IN); Latchumanan Vinoth Kumar, New Delhi (IN); Venkataswamy Manivel, New Delhi (IN); Jabeen Ahmad, New Delhi (IN); Anil Chatterji, Goa (IN); Z. A. Ansari, Goa (IN)

Correspondence Address: Thomas T. Moga, Dickinson Wright PLLC, 1901 L Street NW, Suite 800, Washington, DC, 20036, US

(21) Appl. No.: 10/401,622

(22) Filed: Mar. 28, 2003

Related U.S. Application Data
(60) Provisional application No. 60/367,714, filed on Mar. 28, 2002.

(51) Int. Cl. C12N 5/08; C12N 5/06; A61K 35/56

(52) U.S. Cl. 435/372; 424/93.7; 424/547; 435/354

(57) ABSTRACT

The present invention particularly relates to an extract from Indian green mussel (Pema viridis) which induces differentiation and maturation of dendritic cells wherein said dendritic cells can be used in vaccination, cancer therapy and other immunomodulatory regiments.

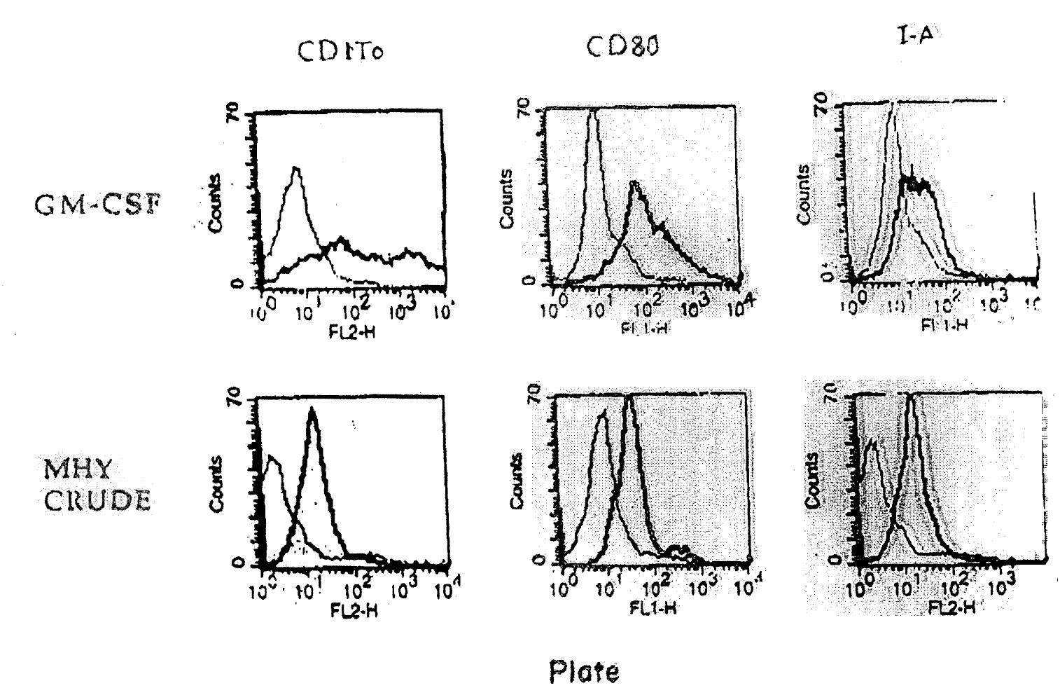

### FIELD OF INVENTION

The present invention particularly relates to an extract from Indian green mussel (Pema viridis) which induces differentiation and maturation of dendritic cells. Said dendritic cells can be used in vaccination, cancer therapy and other immunomodulatory regiments. The extract prepared from the Indian green mussel has been found to possess not only prophylactic efficacy for protection from several viral diseases but it also shows a high therapeutic property against these diseases. The process of preparation of extract was developed for the first time by the Russian scientists. A patent on the process developed by Russian scientists was also filed (Patent No. RU 2043109). Two process patents on extraction of mussel hydrolysate have also been filed by National Institute of Oceanography, Goa, India.

### BACKGROUND OF INVENTION

The mussel hydrolysate has got immuno-modulation properties (Bichurina et a/., 1994). Basically, Immuno-suppression is a common clinical feature in many infectious diseases. This immuno-suppression cripples the ability of the immune systems to get rid of the infectious agent eventuating in the death of the host. It has been found that the host recovering from disease after chemotherapy shows a significant improvement of antigen- specific immune functions. Despite the urgency of the need, and immuno-potentiator which can be of human use remains awaited. The cumulative incidence of the diseases obviates the need for a drug (s) that restores the immune response of the affected individual to a normal level.

In response to any invasion by a pathogen, the complex multicellular organisms have evolved a defense mechanism to make their internal environment more hostile to invaders. All immune systems have one feature in common: they respond to infection by switching from a resting to an active state. The innate response limits the infection and activates Antigen Presenting Cells (APCs) to trigger adaptive immunity, which increases specificity and generates memory. Consequently, the immune responses that occur during an encounter with antigens-be it infectious agents or allergens are primarily characterized by the plasticity of their nature and magnitude. This feature provides an important advantage that permits the immune system to tailor its defense strategy to particular groups of antigens. Interactions between APCs such as dendritic cells (DCs) and macrophages and the different subsets of T cells (CD4⁺ and CD8⁺) in the T cell rich areas of the lymph nodes and spleen, amplify the consequent immune responses. Following this interaction, T helper (Th) cell precursors then differentiate into effectors that secrete either pro-inflammatory or suppressor/regulatory cytokines such as Interferon (IFN)-a, Tumor Necrosis Factor (TNF)-α, and Interleukin (IL)-1, or IL-4, IL-10 and Transforming Growth Factor (TGF)-α, respectively.

Among the most potent of the APCs are the macrophages and different subsets of DCs, that together virtually regulate the antigen capture and presentation of the innate arm of the immune system. DCs are professional APCs that are continuously produced by the stem cells in the hematopoeitic tissues. DCs exist at various states of activation and are primarily classified as immature (iDCs) and mature (mDCs). iDCs are programmed for antigen capture, which upon contact with various stimuli, such as bacterial products, CD40 ligand, (TNF)-α and certain antigens undergo a process of maturation wherein they now become programmed for antigen presentation and T-cell stimulation. Consequently, agents that promote the maturation of iDCs play a vital role in shaping the early immune responses elicited during an infection. Along with macrophages, that constitute the all important phagocytic component of the innate immune system DCs activate the effector cells such as the various subsets of T-cells, Natural Killer (NK) cells and NK-T cells by secreting a profile of cytokines that would eventually prime these effector cells to carryout their functions. This range from stimulating the adaptive arm of the immune system for the generation of antibody mediated responses, to stimulation of cytotoxic activity by the CD8⁺ T cells against the infected cells/tissues.

Therefore, agents that cause the activation of these DCs play a vital role in shaping the character of the immune responses induces early in an infection.

Reference may be made to a patent wherein a recombinant protein such as Granulocyte Macrophage Colony Stimulator Factor (GM/CSF) was used to differentiate and mature the dendritic cells (Rogers, George E., 1991, Eukaryotic plasmid vector encoding enzyme for cysteine production: U.S. Pat. No. 05360742).

The drawback of the above invention is the high cost of the factor (approxt. $51.24 per ug) as compared to the one invented by us which will be 300 times less than GM/CSF.

### OBJECTS OF THE INVENTION

The main objective of the present invention is thus to evaluate an extract prepared from the Indian green mussels (Perna viridis) for inducing differentiation and maturation of dendritic cells. This extract will be useful for inducing differentiation and maturation of dendritic cells. These dendritic cells can be used in vaccination, cancer therapy and other immunomodulatory regiments.

### SUMMARY OF THE INVENTION

The present invention includes extraction of mussel hydrolysate from the Indian green mussels (Perna viridis), collection of bone marrow cells, neutralization of the crude extract, differentiation and maturation of dendritic cells using FACS analysis with florescence markers to see their up-regulation.

### DETAILED DESCRIPTION OF THE INVENTION

Accordingly, the present invention relates to an extract prepared from the Indian green mussels (Perna viridis) which induces differentiation and maturation of dendritic cells. This extract will be useful as a factor in inducing differentiation and maturation of dendritic cells. Such dendritic cells can be used in vaccination, cancer therapy and other immunomodulatory regiments.

In an embodiment, the invention provides an extract prepared from the Indian green mussels (Perna viridis).

In an embodiment, a group of 4 Balb/c mice are sacrificed by transferring the animals in a chloroform chamber, removing the hind limbs carefully and placing them in a petridish with HBSS (Hanks Balanced Saline Solution) wash buffer.

In an embodiment, the bones are scrapped to remove all tissue and chipping of the ends of the bone.

In an embodiment, the bone marrow is extracted by injecting the HBBS solution into the bone with the help of a hypodermal syringe (No gauge).

In an embodiment, the bone marrow is made into a fine suspension by syringing in and out of the fluid several times.

In an embodiment, the suspension is transferred into a 50 ml sterilized centrifuge tubes and the suspension is then centrifuged for 10 minutes.

In an embodiment, the supernatant is removed, RBC lysis buffer is added, mixed the solution thoroughly and incubated for 3 minutes at room temperature.

In an embodiment, the pellets are washed using HBSS wash buffer and then centrifuged again for 10 minutes at.

In an embodiment, the pellet is dissolved and passed through a pre separation filter (Miltenye Biotech # 130-041-407) to remove unwanted tissue.

In an embodiment, the microbeads (I-A, CD45 and CD90) are added to this clear solution and incubated for one hour at 4° C. with shaking to eliminate other lymphocytes and macrophages.

In an embodiment, the suspension is then finally passed through a MACS column to get essentially and predominately dendritic cells.

In an embodiment, the pellets are suspended in complete medium (RPMI 1640, 10% Foetal Calf Serum, Sodium pyruvate and β-Mercaptoethanol.

In an embodiment, about 2.5-3×10⁶ dendritic cells are plated in each well of 24 well culture plate.

In an embodiment, in each of the well of multiwell plate, 50 μl crude extract from Indian green mussel (neutralized with 1 M Tris HCI to achieve a pH of 7.0) is transferred.

In an embodiment, the plate is incubated for 48 hours at 37° C. in an incubator.

In an embodiment, the evaluation of up regulation of various markers associated with dendritic cell differentiation caused by crude green mussel extract is carried out by flow cytometry using fluorescent antibodies to various markers (CD11c, I-A, B-7.1, B7.2, H2L, CD54).

The present invention is further described with respect to the following examples which are given by way of illustration and hence, should not be construed to limit the scope of the invention in any manner.

Extraction of Mussel Hydrolysate

Samples of live bivalves, green mussel (Perna viridis) are collected directly from the sea. The process involved cleaning of green mussels, deshelling and removal of meat and mantle fluid, fermentation, distillation and digestion with concentrated hydrochloric acid, cooling the resultant solution at room temperature and maintaining the pH of the solution with an alkali, isolation of active extract by keeping the resultant solution in a separating flask and carefully removing the middle part of the solution.

Isolation of Bone Marrow

For each set of experiment a group of 4 Balb/c mice were sacrificed by transferring the animals in a chloroform chamber. The hind limbs of the mouse were removed carefully and placed in a petridish with HBSS (Hanks Balanced Saline Solution) wash buffer. The bones were scrapped to remove all tissue attached on it. This was followed by chipping of the ends of bones. The bone marrow was extracted by injecting the HBBS solution into the bone with the help of a hypodermal syringe (No gauge). The bone marrow was finally made into a fine suspension by syringing in and out of the fluid several times.

Differentiation of Dendritic Cells

The fine suspension was transferred into a 50 ml sterilized centrifuge tubes and centrifuged for 10 minutes. After removing the supernatant, RBC lysis buffer was added, mixed thoroughly and incubated for 3 minutes at room temperature. The pellets were washed using HBSS wash buffer and then centrifuged again for 10 minutes. This process was repeated for one more time. The pellet was dissolved and passed through a pre separation filter (Miltenye Biotech # ISO-041-407) to remove unwanted tissue etc. Microbeads (I-A, CD45 and CD90) were added to this clear solution and incubated for one hour at 4° C. with shaking to eliminate other lymphocytes and macrophages. This suspension was then finally passed through a MACS column to get essentially and predominately dendritic cells. The pellets were suspended in complete medium (RPMI 1640, 10% Foetal Calf Serum, Sodium pyruvate and p-Mercaptoethanol). About 2.5-3×10⁶ dendritic cells were plated in each well of a 24 well culture plate. In each of the well of multiwell plate, 50 μl crude extract from Indian green mussel (neutralized with 1 M Tris HCI to achieve a pH of 7.0) was transferred. The plate was incubated for 48 hours at 37° C. in an incubator.

Evaluation of Up Regulation of Markers and Maturation of Dendric Cells

In order to evaluate the up regulation of various markers associated with dendritic cell differentiation caused by crude green mussel extract, flow cytometry was carried out using fluorescent antibodies to various markers (CD11C, I-A, B-7.1, B7.2, H2L, CD54).

The following examples are given by way of illustration of the present invention and therefore, should not be construed to limit the scope of the present invention. The following experiments were conducted several times with reproducible results.

### EXPERIMENT 1

For each set of experiment a group of 4 Balb/c mice were sacrificed by transferring the animals in a chloroform chamber. The hind limbs of the mouse were removed carefully and placed in a petridish with HBSS (Hanks Balanced Saline Solution) wash buffer. The bones were scrapped to remove all tissue attached on it. This was followed by chipping of the ends of bones. The bone marrow was extracted by injecting the HBBS solution into the bone with the help of a hypodermal syringe (No gauge). The bone marrow was finally made into a fine suspension by syringing in and out of the fluid several times. The fine suspension was transferred into 50 ml sterilized centrifuge tubes. The suspension was then centrifuged for 10 minutes at______ rpm. After removing the supernatant, RBC lysis buffer was added, mixed thoroughly and incubated for 3 minutes at room temperature. The pellets were washed using HBSS wash buffer and then centrifuged again for 10 minutes at______ rpm. This process was repeated for one more time. The pellet was dissolvedand passed through a pre separation filter (Miltenye Biotech # 130-041-407) to remove unwanted tissue etc. Microbeads (I-A, CD45 and CD90) were added to this clear solution and incubated for one hour at 4° C. with shaking to eliminate other lymphocytes and macrophages. This suspension was then finally passed through a MACS column to get essentially and predominately dendritic cells. The pellets were suspended in complete medium (RPMI 1640, 10% Foetal Calf Serum, Sodium pyruvate and p-Mercaptoethanol). About 2.5-3×10⁶ dendritic cells were plated in each well of a 24 well culture plate. In each of the well of multiwell plate, 50 μl crude extract from Indian green mussel (neutralized with 1 M Tris HCI to achieve a pH of 7.0) was transferred. The plate was incubated for 48 hours at 37° C. in an incubator.

In order to evaluate the up regulation of various markers associated with dendritic cell differentiation caused by crude green mussel extract, flow cytometry was carried out using fluorescent antibodies to various markers (CD11c, I-A, B-7.1, B7.2, H2L, CD54). The results showed a significant fluorescent shift as compared to the control suggesting the differentiation of dendritic cell (Plate 1).

### THE MAIN ADVANTAGES OF THE PRESENT INVENTION ARE

The extract from the Indian green mussels (Perna viridis) induced the differentiation and maturation of dendritic cells.

The extract from the Indian green mussels (Perna viridis) is 300 times cheaper as compared to the one developed earlier i.e. Granulocyte Macrophage Colony Stimulating factor (GM/CSF).

The invention provides a novel extract which will be useful in vaccination, cancer therapy and other immunomodulatory regiments.

1. A process for inducing differentiation and maturation of dendritic cells useful as antigen presenting cells (APC) in response to immunological disorders, said process comprising the step of adding an effective amount of an extract obtained from Indian green mussels to the dendritic cells and incubating the mixture for a time period ranging between 24 to 72 hours at a temperature range of 30 to 40° C.

2. A process as claimed in claim 1, wherein the mixture is incubated for a time period of 48 hours.

3. A process as claimed in claim 1, wherein the mixture is incubated at a temperature of 35° C.

4. A process as claimed in claim 1, wherein the 2.5 to 3×10⁶ dendritic cells are taken in a well culture plate.

5. A process as claimed in claim 1, wherein 50 μl of the extract is added to the dendric cells in the well culture plate.

6. A process as claimed in claim 1, wherein the extract has a pH of 7.0.

7. A process as claimed in claim 1, wherein the green mussel used is Perna viridis.

8. A process as claimed in claim 1, wherein the dendritic cells are obtained from mice.

9. A process as claimed in claim 1, wherein differentiation and maturation of dendritic cells includes up regulation of the markers.

10. A process as claimed in claim 9, wherein the markers include CD11c, 1A, B-7.1, B-7.2, H2L and CD54.

11. A method for treating immunological disorders in a subject using an extract obtained from Indian green mussel or a composition containing effective amount of said extract with pharamaceutically acceptable carriers, said method comprising administering a pharmaceutically effective amount of said extract or the composition to the subject suffering from immunological disorders.

12. A method as claimed in claim 11, wherein the carrier is selected from nutrients and/or pharmaceutically acceptable carrier, excipient, diluent or solvent.

13. A method as claimed in claim 11, wherein the extract or the composition is administered orally or subcutaneously.

14. A method as claimed in claim 11, wherein the immunological disorder includes cancer.

15. A method as claimed in claim 11, wherein the subject includes animals including human being.

16. A method for inducing up regulation of immune response in a subject, said process comprising administering pharmaceutically effective amount of an extract obtained from Indian green mussel or a composition containing effective amount of said extract with pharamaceutically acceptable carriers to said subject.

17. A method as claimed in claim 16, wherein the carrier is selected from nutrients and/or pharmaceutically acceptable carrier, excipient, diluent or solvent.

18. A method as claimed in claim 16, wherein the extract or the composition is administered orally or subcutaneously.

19. A method as claimed in claim 20, wherein the immunological disorder includes cancer.

20. A method as claimed in claim 20, wherein the subject includes animals including human being.
